(19) United States
(12) Patent Application Publication
(10) Pub. No.: US 2023/0309844 A1
(43) Pub. Date: Oct. 5, 2023

(54) APPARATUS AND METHOD FOR ESTIMATING BLOOD PRESSURE

(71) Applicant: SAMSUNG ELECTRONICS CO., LTD., Suwon-si (KR)

(72) Inventors: Dae Geun JANG, Yongin-si (KR); Ui Kun Kwon, Hwaseong-si (KR); Chang Soon Park, Chungju-si (KR); Young Soo Kim, Seoul (KR)

(73) Assignee: SAMSUNG ELECTRONICS CO., LTD., Suwon-si (KR)

(21) Appl. No.: 17/893,521

(22) Filed: Aug. 23, 2022

(30) Foreign Application Priority Data
Mar. 31, 2022 (KR) 10-2022-0040371

(51) Int. Cl. A61B 5/024; A61B 5/021; A61B 5/00

(52) U.S. Cl. CPC A61B 5/02416; A61B 5/02108; A61B 5/7235; A61B 5/7203

(57) ABSTRACT

An apparatus and method for estimating blood pressure are provided. The apparatus for estimating blood pressure includes: a photoplethysmogram (PPG) sensor configured to measure a PPG signal from an object; and a processor configured to: obtain a blood pressure variation for each of a plurality of blood pressure estimation models based on the PPG signal by using the plurality of blood pressure estimation models, obtain a combining coefficient for each of the plurality of blood pressure estimation models based on the obtained blood pressure variations, and estimate blood pressure by using the obtained combining coefficients.

### CROSS-REFERENCE TO RELATED APPLICATION(S)

This application claims priority from Korean Patent Application No. 10-2022-0040371, filed on Mar. 31, 2022, in the Korean Intellectual Property Office, the disclosure of which is incorporated by reference herein in its entirety.

### BACKGROUND

### 1. Field

Example embodiments of the disclosure relate to an apparatus and method for non-invasively estimating blood pressure by using a photoplethysmogram (PPG) signal.

### 2. Description of Related Art

Research on information technology (IT)-medical convergence technology, in which IT and medical technology are combined, is being recently carried out to address the aging population structure, rapid increase in medical expenses, and shortage of specialized medical service personnel. Particularly, monitoring of the health condition of the human body is not limited to a fixed place, such as a hospital, but is expanding to a mobile healthcare sector for monitoring a user's health status at any time and any place in daily life at home and office. Electrocardiography (ECG), photoplethysmogram (PPG), and electromyography (EMG) signals are examples of bio-signals that indicate the individual's health condition. A variety of signal sensors are being developed to measure such signals in daily life. Particularly, in the case of a PPG sensor, it is possible to estimate blood pressure of a human body by analyzing a pulse wave form that reflects cardiovascular status.

### SUMMARY

According to an aspect of an example embodiment, an apparatus for estimating blood pressure, includes: a photoplethysmogram (PPG) sensor configured to measure a PPG signal from an object; and a processor configured to: obtain a blood pressure variation for each of a plurality of blood pressure estimation models based on the PPG signal by using the plurality of blood pressure estimation models, obtain a combining coefficient for each of the plurality of blood pressure estimation models based on the obtained blood pressure variations, and estimate blood pressure by using the obtained combining coefficients.

The processor may be further configured to: obtain a difference between a reference value and each of the blood pressure variations for each of the plurality of blood pressure estimation models, and obtain the combining coefficient for each of the plurality of blood pressure estimation models based on the obtained difference.

The difference may include at least one of an absolute value of a value, obtained by subtracting the reference value from an absolute value of the blood pressure variation for each of the plurality of blood pressure estimation models, or a Euclidean distance between the absolute value of the blood pressure variation and the reference value.

The processor may be further configured to obtain, as the combining coefficient for each of the plurality of blood pressure estimation models, a value obtained by dividing the difference between the reference value and the blood pressure variation for each of the plurality of blood pressure estimation models by a sum of differences between the reference value and each of the blood pressure variations for the plurality of blood pressure estimation models.

The processor may be further configured to: obtain a final blood pressure variation by applying the combining coefficient for each of the plurality of blood pressure estimation models to each of the corresponding blood pressure variations and by linearly combining the blood pressure variations, and estimate the blood pressure by adding a reference blood pressure to the final blood pressure variation.

The processor may be further configured to: select at least a portion of the plurality of blood pressure estimation models based on the combining coefficient obtained for each of the plurality of blood pressure estimation models, obtain a final blood pressure variation based on blood pressure variations of the selected portion of the plurality of blood pressure estimation models, and estimate the blood pressure based on the final blood pressure variation.

The processor may be further configured to select blood pressure estimation models having combining coefficients greater than or equal to a predetermined threshold value.

The processor may be further configured to obtain, as the final blood pressure variation, a statistical value including a mean value or a median value of the blood pressure variations of the selected portion of the plurality of blood pressure estimation models.

The processor may be further configured to: calculate a statistical value including a mean or standard deviation of the obtained blood pressure variations, and obtain the combining coefficient for each of the plurality of blood pressure estimation models based on the calculated statistical value.

The processor may be further configured to: based on the statistical value being greater than a first predetermined value, determine a high combining coefficient for a blood pressure estimation model having a blood pressure variation above a second predetermined value, and based on the statistical value being less than the first predetermined value, determine a high combining coefficient for a blood pressure estimation model having a blood pressure variation below the second predetermined value.

The processor may be further configured to: divide a plurality of training data into a plurality of training data groups according to the blood pressure variations, and generate blood pressure estimation models for each of the divided training data groups.

According to an aspect of an example embodiment, a method of estimating blood pressure, includes: measuring a photoplethysmogram (PPG) signal from an object; obtaining a blood pressure variation for each of a plurality of blood pressure estimation models based on the PPG signal by using the plurality of blood pressure estimation models; obtaining a combining coefficient for each of the plurality of blood pressure estimation models based on the obtained blood pressure variations; and estimating blood pressure by using the obtained combining coefficients.

The obtaining the combining coefficient for each of the plurality of blood pressure estimation models may include: obtaining a difference between a reference value and the blood pressure variation for each of the plurality of blood pressure estimation models; and obtaining the combining coefficient for each of the plurality of blood pressure estimation models based on the obtained difference.

The obtaining the combining coefficient for each of the plurality of blood pressure estimation models may include obtaining, as the combining coefficient for each of the plurality of blood pressure estimation models, a value obtained by dividing the difference between the reference value and the blood pressure variation for each of the plurality of blood pressure estimation models by a sum of differences between the reference value and each of the blood pressure variations for the plurality of blood pressure estimation models.

The estimating the blood pressure may include: obtaining a final blood pressure variation by applying the combining coefficient for each of the plurality of blood pressure estimation models to each of the corresponding blood pressure variations and linearly combining the blood pressure variations; and adding a reference blood pressure to the final blood pressure variation.

The estimating the blood pressure may include: selecting at least a portion of the plurality of blood pressure estimation models based on the combining coefficients obtained for each of the plurality of blood pressure estimation models; obtaining a final blood pressure variation based on blood pressure variations of the selected portion of the plurality of blood pressure estimation models; and estimating the blood pressure based on the final blood pressure variation.

The selecting at least the portion of the plurality of blood pressure estimation models may include selecting blood pressure estimation models having combining coefficients greater than or equal to a predetermined threshold value.

The estimating the blood pressure may include obtaining, as the final blood pressure variation, a statistical value including a mean value or a median value of the blood pressure variations of the selected portion of the plurality of blood pressure estimation models.

The obtaining the combining coefficient for each of the plurality of blood pressure estimation models may include: calculating a statistical value including a mean or standard deviation of the obtained blood pressure variations; and obtaining the combining coefficient for each of the plurality of blood pressure estimation models based on the calculated statistical value.

According to an aspect of an example embodiment, an electronic device includes: a main body; a photoplethysmogram (PPG) sensor configured to measure a PPG signal from an object; and a processor disposed in the main body, the processor being configured to: obtain a blood pressure variation for each of a plurality of blood pressure estimation models based on the PPG signal by using the plurality of blood pressure estimation models, obtain a combining coefficient for each of the plurality of blood pressure estimation models based on the obtained blood pressure variations, and estimate blood pressure by using the obtained combining coefficients.

### BRIEF DESCRIPTION OF THE DRAWINGS

The above and other aspects, features, and advantages of certain example embodiments of the present disclosure will be more apparent from the following description taken in conjunction with the accompanying drawings, in which:

FIG. 1 is a block diagram illustrating an apparatus for estimating blood pressure according to an example embodiment of the present disclosure.

FIG. 2 is a diagram explaining the principle of generating component waveforms included in unit waveforms of a PPG signal.

FIGS. 3A, 3B, and 3C are block diagrams illustrating a configuration of a processor according to example embodiments of the present disclosure.

FIG. 4 is a block diagram illustrating an apparatus for estimating blood pressure according to another example embodiment of the present disclosure.

FIG. 5 is a flowchart illustrating a method of estimating blood pressure according to an example embodiment of the present disclosure.

FIG. 6 is a flowchart illustrating a method of estimating blood pressure according to another example embodiment of the present disclosure.

FIG. 7 is a flowchart illustrating a method of estimating blood pressure according to yet another example embodiment of the present disclosure.

FIGS. 8, 9, and 10 are block diagrams illustrating various structures of an electronic device including an apparatus for estimating blood pressure.

### DETAILED DESCRIPTION

Example embodiments are included in the following detailed description and drawings. Advantages and features of the present invention, and a method of achieving the same will be more clearly understood from the following example embodiments described in detail with reference to the accompanying drawings. Throughout the drawings and the detailed description, unless otherwise described, the same drawing reference numerals will be understood to refer to the same elements, features, and structures. The relative size and depiction of these elements may be exaggerated for clarity, illustration, and convenience.

It will be understood that, although the terms first, second, etc. may be used herein to describe various elements, these elements should not be limited by these terms. These terms are only used to distinguish one element from another. Any references to singular may include plural unless expressly stated otherwise. In addition, unless explicitly described to the contrary, an expression such as “comprising” or “including” will be understood to imply the inclusion of stated elements but not the exclusion of any other elements. Also, the terms, such as “unit” or “module”, etc., should be understood as a unit for performing at least one function or operation and that may be embodied as hardware, software, or a combination thereof.

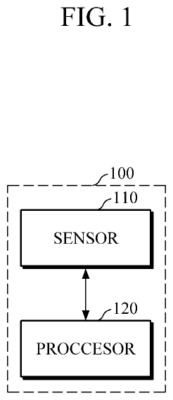

FIG. 1 is a block diagram illustrating an apparatus for estimating blood pressure according to an example embodiment of the present disclosure.

Various examples of the apparatus for estimating blood pressure may be included in an electronic device, such as a smartphone, a tablet PC, a desktop computer, a laptop computer, or a wearable device including a wristwatch-type wearable device, a bracelet-type wearable device, a wristband-type wearable device, a ring-type wearable device, a glasses-type wearable device, a headband-type wearable device, and the like.

Referring to FIG. 1, an apparatus 100 for estimating blood pressure includes a sensor 110 and a processor 120.

The sensor 110 may measure a bio-signal from an object. For example, the bio-signal may include photoplethysmogram (PPG), Electrocardiography (ECG), Electromyography (EMG), impedance plethysmogram (IPG), pressure wave, video plethysmogram (VPG), and other data indicating physiological or pathological information. In some embodiments, the object may be a body part that comes into contact with or is adjacent to the sensor 110, and may be a body part where pulse waves may be easily measured. For example, the object may be a skin area of the wrist that is adjacent to the radial artery and a skin area of the human body where venous blood or capillary blood passes. However, the object is not limited thereto, and may be a peripheral part of the body, such as a finger, a toe, or the like, where blood vessels are densely distributed in the human body.

For example, the sensor 110 may include a PPG sensor for measuring a PPG signal from an object, and the PPG sensor may include one or more light sources for emitting light toward the object and one or more detectors for detecting light scattered or reflected from or transmitted through the object after light is emitted by the light sources. In this case, the light source may include a light emitting diode (LED), a laser diode (LD), a phosphor, and the like. The light sources may emit light of one or more wavelengths (e.g., green, red, blue, and infrared wavelengths). In addition, the detectors may include one or more photodiodes, photo transistors (PTr), image sensors (e.g., complementary metal-oxide semiconductor (CMOS) image sensor), etc., but is not limited thereto.

The processor 120 may be electrically or functionally connected to the sensor 110 and may control the sensor 110 to acquire a bio-signal. Upon receiving the bio-signal, the processor 120 may perform preprocessing, such as removing noise from the received bio-signal. For example, the processor 120 may perform signal correction, such as filtering (e.g., band-pass filtering between 0.4 Hz and 10 Hz), amplification of the bio-signal, converting the signal into a digital signal, smoothing, ensemble averaging of a continuously measured pulse wave signal, and other medically useful data manipulations. In addition, the processor 120 may obtain a plurality of unit waveforms by segmenting a waveform of a bio-signal, measured continuously during a predetermined period of time, into cycles, and may determine a representative waveform for use in estimating blood pressure by using any one or a combination of two or more of the plurality of unit waveforms.

By using a plurality of predefined blood pressure estimation models, the processor 120 may estimate blood pressure based on the bio-signal measured by the sensor 110. The plurality of blood pressure estimation models may estimate blood pressure variations relative to blood pressure at a calibration time. In this case, the blood pressure variations may refer to variations in mean arterial pressure (MAP), diastolic blood pressure (DBP), or systolic blood pressure (SBP).

For example, by inputting the PPG signal to the respective blood pressure estimation models, the processor 120 may obtain blood pressure variations for the respective blood pressure estimation models. In addition, upon obtaining the blood pressure variations for the respective blood pressure estimation models, the processor 120 may obtain combining coefficients for the respective blood pressure estimation models based on magnitudes of the obtained blood pressure variations, and may estimate blood pressure by using the obtained combining coefficients.

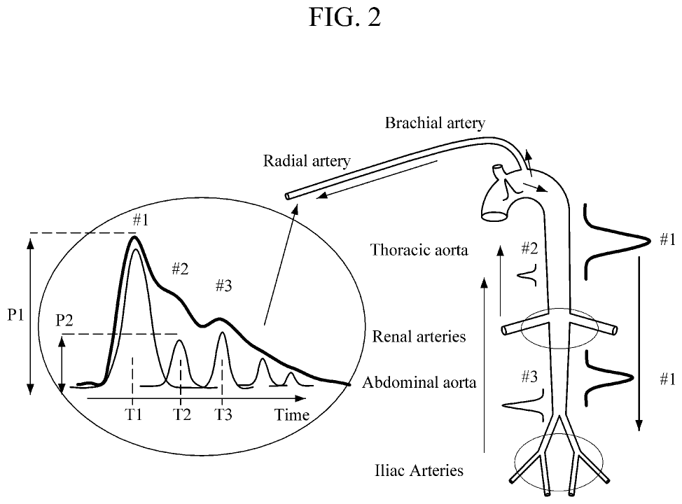

FIG. 2 is a diagram explaining the principle of generating component waveforms included in unit waveforms of a PPG signal.

Referring to FIG. 2, a PPG signal may be generally a summation of a propagation wave #1 propagating from the heart by blood ejection from the left ventricle to peripheral parts of the body or branching points in the blood vessels, and reflection waves #2 and #3 returning from the peripheral parts of the body or the branching points in the blood vessels. The propagation wave #1 is related to cardiac characteristics, and the reflection waves #2 and #3 are related to vascular characteristics. Generally, the propagation wave #1 generated by blood ejection from the left ventricle is mainly reflected from the renal arteries and iliac arteries, to generate the first reflection wave #2 and the second reflection wave #3. As described above, by dividing the unit waveforms of the pulse wave signal into the respective component waveforms #1, #2, and #3, and by analyzing time points T1, T2, and T3 associated with the component waveforms #1, #2, and #3 and/or amplitudes P1, P2, and P3 of the pulse wave signal, etc., the processor 120 may estimate blood pressure.

Generally, a variation in MAP is generally proportional to cardiac output (CO) and total peripheral resistance (TPR), as shown in the following Equation 1.

ΔMAP=CO×TPR [Equation 1]

Herein, ΔMAP denotes a difference in MAP between the left ventricle and the right atrium, in which MAP of the right atrium is generally in a range of 3 mmHg to 5 mmHg, such that the MAP in the right atrium is similar to the MAP in the left ventricle or MAP of the upper arm. If absolute actual cardiac output (CO) and total peripheral resistance (TPR) values are known, MAP may be obtained from the aorta or the upper arm. However, it may be difficult to estimate absolute values of CO and TPR based on a PPG signal. Accordingly, by properly using a CO feature and/or the TPR feature for each blood pressure estimation model, the blood pressure variations may be obtained, and a final blood pressure value may be obtained by combining the respective blood pressure variations. Here, the CO feature may be a feature value which shows an increasing/decreasing trend in proportion to an actual CO value which relatively increases/decreases when an actual TPR value does not change significantly compared to resting total peripheral resistance. Further, the TPR feature may be a feature value which shows an increasing/decreasing trend in proportion to an actual TPR value which relatively increases/decreases when an actual CO value does not change significantly compared to resting cardiac output.

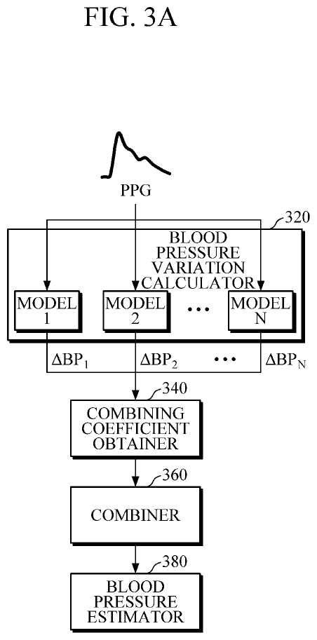

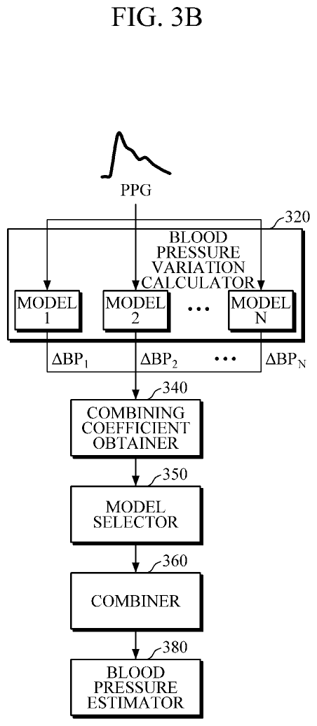

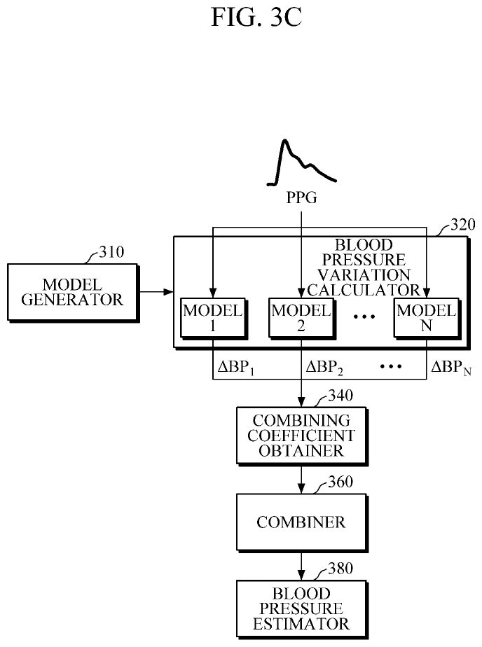

Hereinafter, various examples of estimating blood pressure by the processor 120 will be described with reference to FIGS. 3A, 3B, and 3C.

FIG. 3A is a block diagram illustrating a configuration of a processor according to an example embodiment of the present disclosure.

Referring to FIG. 3A, the processor 120 according to an example embodiment may include a blood pressure variation calculator 320, a combining coefficient obtainer 340, a combiner 360, and a blood pressure estimator 380.

The blood pressure variation calculator 320 may preprocess a PPG signal, and by inputting, for example, a representative waveform of the PPG signal, which is generated as a result of the preprocessing, to each of blood pressure estimation models (model 1, model 2, . . . , and model N), the blood pressure variation calculator 320 may calculate blood pressure variations for the respective blood pressure estimation models.

The respective blood pressure estimation models may obtain required features by analyzing the representative waveform of the PPG signal, and may output blood pressure variations by using the obtained features. For example, the respective blood pressure estimation models may extract a variety of information by analyzing the representative waveform, and may obtain the features by combining the extracted information. In this case, information items extracted from the PPG signal may include heart rate (HR), a time T1 and/or an amplitude P1 of the propagation wave #1 of the above component waveform, a time T2 and/or an amplitude P2 of the reflection wave #2 thereof, a time and/or an amplitude at a maximum point in a predetermined region (e.g., systolic phase) of the PPG signal, a time and/or an amplitude at a point where a slope is closest to zero in a predetermined region, a time and/or an amplitude at an internally dividing point between the time T1 of the propagation wave #1 and the time T2 of the reflection wave #2, a time and/or an amplitude at an internally dividing point between the time T1 of the propagation wave #1 and the time at the point where the slope is closest to zero in the predetermined region, a total or partial area of the PPG signal, a duration of the PPG signal, cycles, information related to the pulse wave, and the like. However, the information is not limited thereto.

The features may be defined differently for each blood pressure estimation model. For example, blood pressure estimation model 1 may be defined to use a ratio P2/P1 between the amplitudes of the reflection wave and the propagation wave, and blood pressure estimation model 2 may be defined to use a ratio Pmax/P1 between an amplitude Pmax at a maximum point in a predetermined region and the amplitude P1 of the propagation wave. In addition, the blood pressure variation calculator 320 may obtain blood pressure variations ΔBP₁, ΔBP₂, . . . , and ΔBP_{N} by inputting the respective features to corresponding blood pressure estimation models.

The combining coefficient obtainer 340 may obtain combining coefficients for the respective blood pressure estimation models based on magnitudes of the blood pressure variations obtained for the respective blood pressure estimation models.

For example, the combining coefficient obtainer 340 may obtain a difference between the blood pressure variation and a reference value for each blood pressure estimation model, and may obtain the combining coefficient for each blood pressure estimation model based on the obtained difference. In this case, the reference value may be predetermined in consideration of characteristics of the blood pressure estimation models. In this case, the difference between the blood pressure variation and the reference value may be defined as an absolute value of a value, obtained by subtracting the reference value from an absolute value of the blood pressure variation, or Euclidean distance between the blood pressure variation and the reference value, and the like. For example, the combining coefficient of a specific blood pressure estimation model may be defined as a value, obtained by dividing a difference between the reference value and the blood pressure variation for the specific blood pressure estimation model by a sum of the differences between the reference value and the blood pressure variations of all the blood pressure estimation models.

The following Equation 2 is an example of obtaining the combining coefficient based on the absolute value of the value obtained by subtracting the reference value from the absolute value of the blood pressure variation.

$${w}_{i} = \frac{\vert \vert {Δ BP}_{esi , i} \vert - {X}_{ref} \vert}{{\sum }_{j = 1}^{N} \vert \vert {Δ BP}_{esi , i} \vert - {X}_{ref} \vert} \qquad [ Equation 2 ]$$

Herein, i denotes an index of the blood pressure estimation models; Wᵢ denotes the combining coefficient of blood pressure estimation model i; ΔBPₑₛₜ, denotes the blood pressure variation output from the blood pressure estimation model i; X_{ref} denotes a predefined reference value; and N denotes the number of blood pressure estimation models.

In another example, the combining coefficient obtainer 340 may obtain a statistical value (e.g., mean, standard deviation, etc.) of the blood pressure variations obtained for the respective blood pressure estimation models, and may obtain the combining coefficients for the respective blood pressure estimation models based on a magnitude of the statistical value. If there is a significant change in blood pressure, a mean or standard deviation of the blood pressure variations output from the plurality of blood pressure estimation models is generally high; and if not, the mean or standard deviation is generally low. Accordingly, if the mean or standard deviation of the blood pressure variations is high, e.g., greater than or equal to a predetermined threshold value, a higher combining coefficient may be applied to a greater blood pressure variation; and by contrast, if the mean or standard deviation of the blood pressure variations is low, e.g., smaller than the predetermined threshold value, a higher combining coefficient may be applied to a smaller blood pressure variation.

By applying the combining coefficients, obtained for the respective blood pressure estimation models, to the respective corresponding blood pressure variations and by combining the resultant blood pressure variations, the combiner 360 may obtain a final blood pressure variation. The following Equation 3 is an example of linear combination, but is not limited thereto and may be defined as various nonlinear combination equations.

$${Δ BP}_{esi , i} = {\sum }_{i = 1}^{N} {w}_{i} \times {Δ BP}_{i} \qquad [ Equation 3 ]$$

Herein, i denotes an index of blood pressure estimation models; N denotes the number of blood pressure estimation models; Wᵢ denotes the combining coefficient of blood pressure estimation model i; ΔBPᵢ denotes the blood pressure variation output from the blood pressure estimation model i; ΔBPₑₛₜ denotes a final blood pressure variation.

In this manner, if it is determined that a blood pressure change is small, a relatively higher combining coefficient may be determined for the blood pressure variation having a small magnitude; and if it is determined that a blood pressure change is large, a relatively higher combining coefficient may be determined for the blood pressure variation having a large magnitude, such that the blood pressure variations may be combined in a manner that is more sensitive to blood pressure changes, thereby estimating blood pressure with improved accuracy.

The blood pressure estimator 380 may estimate a final blood pressure by adding a reference blood pressure to the final blood pressure variation, as shown in the following Equation 4. In this case, the reference blood pressure may be blood pressure obtained at a calibration time before a current estimation time, and may be, for example, blood pressure obtained using a cuff sphygmomanometer and the like.

BPₑₛₜ=ΔBPₑₛₜ+BP_{cal} [Equation 4]

Herein, ΔBPₑₛₜ denotes the final blood pressure variation obtained as described above; BPₑₛₜ denotes the final blood pressure; and BP_{cal} denotes the reference blood pressure at the calibration time.

Referring to FIG. 3B, the processor 120 according to another example embodiment may include the blood pressure variation calculator 320, the combining coefficient obtainer 340, a model selector 350, the combiner 360, and the blood pressure estimator 380. The blood pressure variation calculator 320, the combining coefficient obtainer 340, the combiner 360, and the blood pressure estimator 380 are described above, such that the following description will be focused on non-redundant features.

Once the combining coefficient obtainer 34 obtains the combining coefficients for the respective blood pressure estimation models, the model selector 350 may select valid blood pressure estimation models by using the obtained combining coefficients. For example, the model selector 350 may select blood pressure estimation models, having combining coefficients greater than or equal to a predetermined threshold value, as valid models. In this manner, among the plurality of blood pressure estimation models, if models having high combining coefficients are models defined to estimate a relatively low blood pressure variation, the model selector 350 may determine that a current estimation time of blood pressure is a resting state in which a blood pressure change is small, and may select models defined to estimate a low blood pressure variation as valid estimation models; by contrast, among the plurality of blood pressure estimation models, if models having high combining coefficients are models defined to estimate a relatively high blood pressure variation, the model selector 350 may determine that a current estimation time of blood pressure is a state in which blood pressure changes, and may select models defined to estimate a high blood pressure variation as valid estimation models.

The combiner 360 may obtain a final blood pressure variation by combining the blood pressure variations of the selected valid models. For example, the combiner 360 may obtain a statistical value, e.g., a mean value or a median value, of the blood pressure variations of the selected valid models as the final blood pressure variation. However, the combiner 360 is not limited thereto and may apply the combining coefficients of the selected valid models to corresponding blood pressure variations and then may linearly/nonlinearly combine the resultant blood pressure variations, as described above.

Referring to FIG. 3C, the processor 120 according to yet another example embodiment may include a model generator 310, the blood pressure variation calculator 320, the combining coefficient obtainer 340, the combiner 360, and the blood pressure estimator 380. In addition, the processor 120 may further include the model selector 350 illustrated in FIG. 3B. The blood pressure variation calculator 320, the combining coefficient obtainer 340, the model selector 350, the combiner 360, and the blood pressure estimator 380 are described above, such that the following description will be focused on non-redundant features.

The model generator 310 may collect a plurality of training data, and may generate a plurality of blood pressure estimation models by using the training data. In this case, the training data may include a plurality of PPG signals obtained from a plurality of users during various changes in blood pressure and/or an actually measured blood pressure, or a plurality of PPG signals obtained from a specific user at various blood pressure changing times and/or an actually measured blood pressure. In this case, the actually measured blood pressure may be blood pressure obtained using a cuff sphygmomanometer and the like.

The model generator 310 may divide the plurality of training data into a plurality of training data groups according to a magnitude of blood pressure variation, and may generate a blood pressure estimation model for each group by using the training data of each group. For example, the model generator 310 may divide the data into group 1 in which an absolute value of the blood pressure variation is less than or equal to 5, group 2 in which an absolute value thereof is greater than 5 and less than or equal to 10, . . . , and group N in which an absolute value thereof is greater than 30 and less than or equal to 35. In this case, the blood pressure variation may refer to a difference between the measured blood pressure of each user and a reference blood pressure (e.g., blood pressure obtained at a calibration time for each user). That is, a model trained by using training data of group 1 may be generated to output a relatively low blood pressure variation, and a model trained by using training data of group N may be generated to output a relatively high blood pressure variation, thereby reducing a problem of underestimating blood pressure which occurs when models trained using the same training data estimate blood pressure during changes in blood pressure.

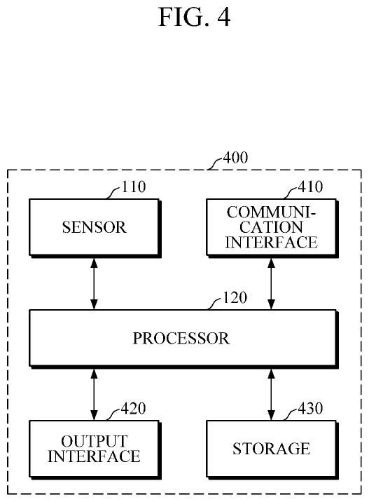

FIG. 4 is a block diagram illustrating an apparatus for estimating blood pressure according to another example embodiment of the present disclosure.

Referring to FIG. 4, an apparatus 400 for estimating blood pressure includes the sensor 110, the processor 120, a communication interface 410, an output interface 420, and a storage 430. The sensor 110 and the processor 120 are described above in detail, and thus a description thereof will be omitted.

The communication interface 410 may be electrically connected to the processor 120, and may communicate with an external electronic device under the control of the processor 120 by using various communication techniques to transmit and receive necessary data, e.g., reference blood pressure, various blood pressure estimation equations, blood pressure estimation results, and the like. The external electronic device may include a blood pressure measuring apparatus, such as a sphygmomanometer, or a smartphone, a tablet PC, a desktop computer, a laptop computer, a wearable device, etc., but is not limited thereto. In this case, the communication techniques may include Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WIFI communication, 3G, 4G, and 5G, communications, and the like. However, the communication techniques are not limited thereto.

The output interface 420 may output processing results of the sensor 110 and/or the processor 120 and may provide the results to a user. The output interface 420 may provide the user with information by various visual/non-visual methods using a visual output module including a display, an audio output module such as a speaker, or a haptic module using vibrations, tactile sensation, and the like.

The storage 430 may store data required for the sensor 110 and/or the processor 120, and/or the processing results of the sensor 110 and/or the processor 120. For example, the storage 430 may store blood pressure estimation equations, criteria for determining reliability, user characteristics (e.g., gender, age, health condition, etc.), the pulse wave signal, features, and reference blood pressure, which are obtained during calibration, and the pulse wave signal, features, and estimated blood pressure values, which are generated at the blood pressure estimation time, and the like.

The storage 430 may include at least one storage medium of a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like, but is not limited thereto.

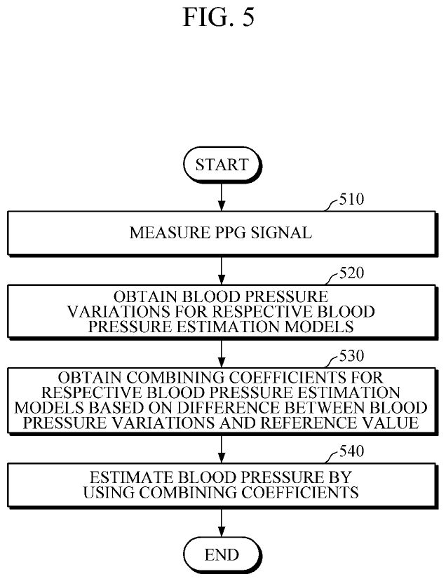

FIG. 5 is a flowchart illustrating a method of estimating blood pressure according to an example embodiment of the present disclosure. The method of FIG. 5 is an example of a method of estimating blood pressure performed by the apparatuses for estimating blood pressure of FIGS. 1 and 4, which are described above in detail, and thus will be briefly described below.

First, the apparatus for estimating blood pressure may measure a PPG signal from an object in 510 in response to a request for estimating blood pressure.

Then, by inputting the PPG signal to a plurality of blood pressure estimation models, the apparatus for estimating blood pressure may obtain blood pressure variations for the respective blood pressure estimation models in 520. The respective blood pressure estimation models may be trained by using different training data sets divided according to the magnitude of the blood pressure variations, and may be models for outputting blood pressure variations relative to blood pressure at a calibration time.

Subsequently, the apparatus for estimating blood pressure may obtain combining coefficients for the respective blood pressure estimation models based on a difference between the blood pressure variations of the respective blood pressure estimation models and a reference value in 530. For example, the difference between the blood pressure variations and the reference value may refer to a value, obtained by subtracting the reference value from an absolute value of the blood pressure variation, or Euclidean distance between the blood pressure variation and the reference value, and the like. For example, the apparatus for estimating blood pressure may obtain, as the combining coefficient of each blood pressure estimation model, a value obtained by dividing the difference between the reference value and the blood pressure variation of each blood pressure estimation model by a sum of the differences between the reference value and the blood pressure variations of all the blood pressure estimation models.

Next, the apparatus for estimating blood pressure may estimate blood pressure by using the obtained combining coefficients in 540. For example, as shown in the above Equation 3, by multiplying the blood pressure variations of the respective blood pressure estimation models by the combining coefficients and by combining the resultant blood pressure variations, the apparatus for estimating blood pressure may obtain a final blood pressure variation. In addition, by adding a reference blood pressure to the obtained final blood pressure variation, the apparatus for estimating blood pressure may obtain a final blood pressure. Upon estimating the blood pressure, the apparatus for estimating blood pressure may output the estimated blood pressure value by using various output means, such as a display, a speaker, a haptic device, etc., and may provide the estimated value to a user.

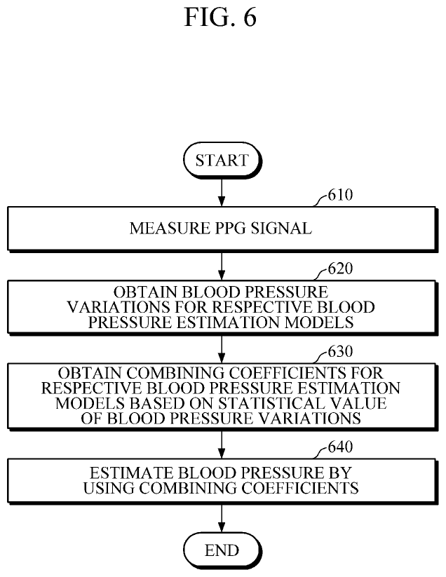

FIG. 6 is a flowchart illustrating a method of estimating blood pressure according to another example embodiment of the present disclosure. The method of FIG. 6 is an example of a method of estimating blood pressure performed by the apparatus for estimating blood pressure of FIG. 1 or FIG. 4, which is described above in detail, and thus will be briefly described below.

First, the apparatus for estimating blood pressure may measure a PPG signal from an object in 610 in response to a request for estimating blood pressure.

Then, by inputting the PPG signal to a plurality of blood pressure estimation models, the apparatus for estimating blood pressure may obtain blood pressure variations for the respective blood pressure estimation models in 620.

Subsequently, the apparatus for estimating blood pressure may obtain combining coefficients for the respective blood pressure estimation models based on a magnitude of a statistical value (e.g., mean, standard deviation, etc.) of the obtained blood pressure variations in 630. For example, if the mean or standard deviation of the blood pressure variations is high, e.g., greater than or equal to a predetermined threshold value, a higher combining coefficient may be applied to a greater blood pressure variation; by contrast, if the mean or standard deviation of the blood pressure variations is low, e.g., smaller than the predetermined threshold value, a higher combining coefficient may be applied to a smaller blood pressure variation.

Next, the apparatus for estimating blood pressure may estimate blood pressure by using the obtained combining coefficients in 640. For example, by applying the combining coefficients of the respective blood pressure estimation models to the respective corresponding blood pressure variations and then linearly combining the blood pressure variations, the apparatus for estimating blood pressure may obtain a final blood pressure variation, and may obtain a final blood pressure by adding a reference blood pressure to the obtained final blood pressure variation. Upon estimating the blood pressure, the apparatus for estimating blood pressure may output the estimated blood pressure value by using various output means, such as a display, a speaker, a haptic device, etc., and may provide the estimated value to a user.

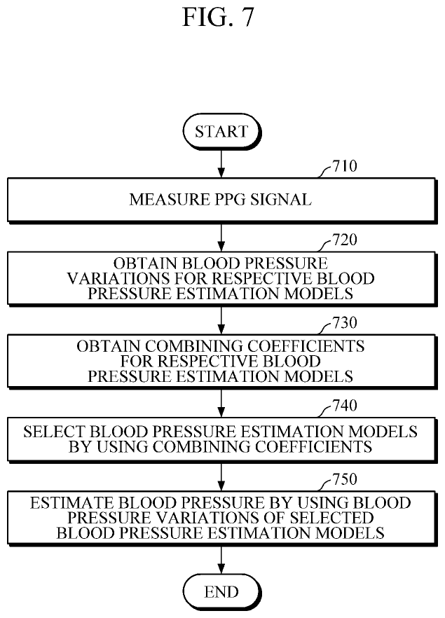

FIG. 7 is a flowchart illustrating a method of estimating blood pressure according to yet another example embodiment of the present disclosure. The method of FIG. 7 is an example of a method of estimating blood pressure performed by the apparatuses for estimating blood pressure of FIGS. 1 and 4, which are described above in detail, and thus will be briefly described below.

First, the apparatus for estimating blood pressure may measure a PPG signal from an object in 710 in response to a request for estimating blood pressure.

Then, by inputting the PPG signal to a plurality of blood pressure estimation models, the apparatus for estimating blood pressure may obtain blood pressure variations for the respective blood pressure estimation models in 720.

Subsequently, the apparatus for estimating blood pressure may obtain combining coefficients for the respective blood pressure estimation models based on the magnitude of the blood pressure variations of the respective blood pressure estimation models in 730. For example, the apparatus for estimating blood pressure may obtain the combining coefficients for the respective blood pressure estimation models based on a difference between the blood pressure variation and the reference value, or a statistical value, such as a mean or standard deviation, of the blood pressure variations, as described above.

Next, the apparatus for estimating blood pressure may select valid blood pressure estimation models by using the obtained combining coefficients in 740. For example, the apparatus for estimating blood pressure may select blood pressure estimation models, having combining coefficients greater than or equal to a predetermined threshold value, as valid blood pressure estimation models. In this manner, in a resting state in which blood pressure is constant, the apparatus for estimating blood pressure may select blood pressure estimation models, defined to estimate a low blood pressure variation (e.g., models trained by using training data sets having a relatively small blood pressure variation), as valid estimation models; by contrast, in a state in which blood pressure changes, the apparatus for estimating blood pressure may select blood pressure estimation models, defined to estimate a high blood pressure variation (e.g., models trained by using training data sets having a relatively large blood pressure variation), as valid estimation models, thereby reflecting a change in blood pressure while blood pressure is currently estimated.

Then, the apparatus for estimating blood pressure may obtain a final blood pressure variation by combining the blood pressure variations of the selected valid models, and may estimate blood pressure by using the final blood pressure variation. For example, the apparatus for estimating blood pressure may obtain a statistical value, e.g., a mean value or a median value, of the blood pressure variations of the selected models as the final blood pressure variation. Upon estimating the blood pressure, the apparatus for estimating blood pressure may output the estimated blood pressure value by using various output means, such as a display, a speaker, a haptic device, etc., and may provide the estimated value to a user.

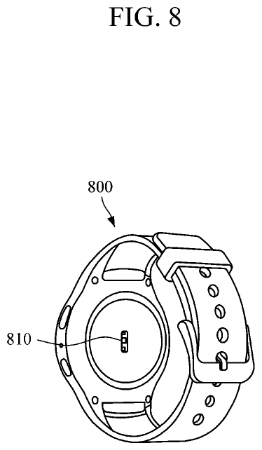

FIGS. 8 to 10 are block diagrams illustrating various structures of an electronic device including the apparatus 100 or 400 for estimating blood pressure of FIG. 1 or FIG. 4.

The electronic device may include, for example, various types of wearable devices, e.g., a smart watch, a smart band, smart glasses, smart earphones, a smart ring, a smart patch, and a smart necklace, and a mobile device such as a smartphone, a tablet PC, etc., or home appliances or various Internet of Things (IoT) devices (e.g., home IoT device, etc.) based on Internet of Things (IoT) technology.

The electronic device may include a sensor device, a processor, an input device, a communication module, a camera module, an output device, a storage device, and a power module. All the components of the electronic device may be integrally mounted in a specific device or may be distributed in two or more devices. The sensor device may include a sensor of the apparatuses 100 and 400 for estimating blood pressure, and may further include an additional sensor, such as a gyro sensor, a Global Positioning System (GPS), and the like.

The processor may execute programs, stored in the storage device, to control components connected to the processor, and may perform various data processing or computation, including estimation of blood pressure. The processor may include a main processor, e.g., a central processing unit (CPU) or an application processor (AP), etc., and an auxiliary processor, e.g., a graphics processing unit (GPU), an image signal processor (ISP), a sensor hub processor, or a communication processor (CP), etc., which is operable independently from, or in conjunction with, the main processor.

The input device may receive a command and/or data to be used by each component of the electronic device, from a user and the like. The input device may include, for example, a microphone, a mouse, a keyboard, or a digital pen (e.g., a stylus pen, etc.).

The communication module may support establishment of a direct (e.g., wired) communication channel and/or a wireless communication channel between the electronic device and other electronic device, a server, or the sensor device within a network environment, and performing of communication via the established communication channel. The communication module may include one or more communication processors that are operable independently from the processor and supports a direct communication and/or a wireless communication. The communication module may include a wireless communication module, e.g., a cellular communication module, a short-range wireless communication module, or a global navigation satellite system (GNSS) communication module, etc., and/or a wired communication module, e.g., a local area network (LAN) communication module, a power line communication (PLC) module, and the like. These various types of communication modules may be integrated into a single chip, or may be separately implemented as multiple chips. The wireless communication module may identify and authenticate the electronic device in a communication network by using subscriber information (e.g., international mobile subscriber identity (IMSI), etc.) stored in a subscriber identification module.

The camera module may capture still images or moving images. The camera module may include a lens assembly having one mor more lenses, image sensors, image signal processors, and/or flashes. The lees assembly included in the camera module may collect light emanating from a subject to be imaged.

The output device may visually/non-visually output data generated or processed by the electronic device. The output device may include a sound output device, a display device, an audio module, and/or a haptic module.

The sound output device may output sound signals to the outside of the electronic device. The sound output device may include a speaker and/or a receiver. The speaker may be used for general purpose, such as playing multimedia or playing record, and the receiver may be used for incoming calls. The receiver may be implemented separately from, or as part of, the speaker.

The display device may visually provide information to the outside of the electronic device. The display device may include, for example, a display, a hologram device, or a projector and control circuitry to control the devices. The display device may include touch circuitry adapted to detect a touch, and/or sensor circuitry pressure sensor, etc.) adapted to measure the intensity of force incurred by the touch.

The audio module may convert a sound into an electrical signal or vice versa. The audio module may obtain the sound via the input device, or may output the sound via the sound output device, and/or a speaker and/or a headphone of another electronic device directly or wirelessly connected to the electronic device.

The haptic module may convert an electrical signal into a mechanical stimulus (e.g., vibration, motion, etc.) or electrical stimulus which may be recognized by a user by tactile sensation or kinesthetic sensation. The haptic module may include, for example, a motor, a piezoelectric element, and/or an electric stimulator.

The storage device may store driving conditions required for driving the sensor device, and various data required for other components of the electronic device. The various data may include, for example, software and input data and/or output data for a command related thereto. The storage device may include a volatile memory and/or a non-volatile memory.

The power module may manage power supplied to the electronic device. The power module may be implemented as part of, for example, a power management integrated circuit (PMIC). The power module may include a battery, which may include a primary cell which is not rechargeable, a secondary cell which is rechargeable, and/or a fuel cell.

Referring to FIG. 8, the electronic device may be implemented as a wristwatch wearable device 800, and may include a main body and a wrist strap. A display is provided on a front surface of the main body, and may display various application screens, including time information, received message information, and the like. A sensor device 810 may be disposed on a rear surface of the main body.

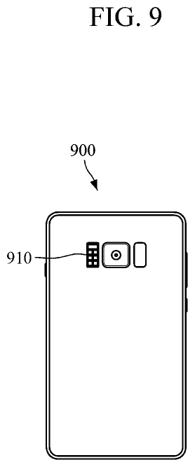

Referring to FIG. 9, the electronic device may be implemented as a mobile device 900 such as a smartphone. The mobile device 900 may include a housing and a display panel. The housing may form an outer appearance of the mobile device 900. The housing has a first surface, on which a display panel and a cover glass may be disposed sequentially, and the display panel may be exposed to the outside through the cover glass. A sensor device 910, a camera module and/or an infrared sensor, and the like may be disposed on a second surface of the housing. The processor and various other components may be disposed in the housing.

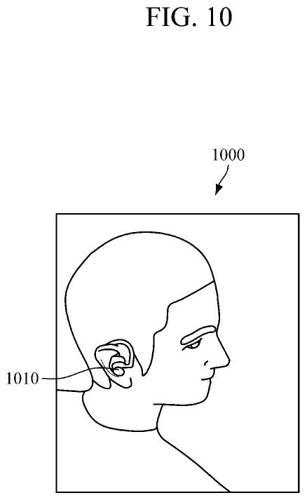

Referring to FIG. 10, the electronic device may be implemented as an ear-wearable device 1000. The ear-wearable device 1000 may include a main body and an ear strap. A user may wear the ear-wearable device 1000 by hanging the ear strap on the auricle. The ear strap may be omitted depending on a shape of the ear-wearable device 1000. The main body may be inserted into the external auditory meatus. A sensor device 1010 may be mounted in the main body. Further, the processor may be disposed in the main body, and may estimate blood pressure by using a PPG signal measured by the sensor device 1010. Alternatively, the ear-wearable device 1000 may estimate blood pressure by interworking with an external device. For example, the ear-wearable device 1000 may transmit the PPG signal, measured by the sensor device 1010 of the ear-wearable device 1000, to an external device, e.g., a smartphone, a tablet PC, etc., through a communication module provided in the main body, so that a processor of the external device may estimate blood pressure, and may output the estimated blood pressure value through a sound output module provided in the main body of the ear-wearable device 1000.

The present disclosure can be realized as a computer-readable code written on a computer-readable recording medium. The computer-readable recording medium may be any type of recording device in which data is stored in a computer-readable manner.

Examples of the computer-readable recording medium include a ROM a RAM, a CD-ROM, a magnetic tape, a floppy disc, an optical data storage, and a carrier wave (e.g., data transmission through the Internet). The computer-readable recording medium can be distributed over a plurality of computer systems connected to a network so that a computer-readable code is written thereto and executed therefrom in a decentralized manner. Functional programs, codes, and code segments needed for realizing the present invention can be readily deduced by programmers of ordinary skill in the art to which the invention pertains.

The present disclosure has been described herein with regard to example embodiments. However, it will be obvious to those skilled in the art that various changes and modifications can be made without changing technical conception and essential features of the present disclosure. Thus, it is clear that the above-described embodiments are illustrative in all aspects and are not intended to limit the present disclosure.

What is claimed is:

1. An apparatus for estimating blood pressure, the apparatus comprising:
a photoplethysmogram (PPG) sensor configured to measure a PPG signal from an object; and
a processor configured to:
obtain a blood pressure variation for each of a plurality of blood pressure estimation models based on the PPG signal by using the plurality of blood pressure estimation models,
obtain a combining coefficient for each of the plurality of blood pressure estimation models based on the obtained blood pressure variations, and
estimate blood pressure by using the obtained combining coefficients.

2. The apparatus of claim 1, wherein the processor is further configured to:
obtain a difference between a reference value and each of the blood pressure variations for each of the plurality of blood pressure estimation models, and
obtain the combining coefficient for each of the plurality of blood pressure estimation models based on the obtained difference.

3. The apparatus of claim 2, wherein the difference comprises at least one of an absolute value of a value, obtained by subtracting the reference value from an absolute value of the blood pressure variation for each of the plurality of blood pressure estimation models, or a Euclidean distance between the absolute value of the blood pressure variation and the reference value.

4. The apparatus of claim 2, wherein the processor is further configured to:
obtain, as the combining coefficient for each of the plurality of blood pressure estimation models, a value obtained by dividing the difference between the reference value and the blood pressure variation for each of the plurality of blood pressure estimation models by a sum of differences between the reference value and each of the blood pressure variations for the plurality of blood pressure estimation models.

5. The apparatus of claim 1, wherein the processor is further configured to:
obtain a final blood pressure variation by applying the combining coefficient for each of the plurality of blood pressure estimation models to each of the corresponding blood pressure variations and by linearly combining the blood pressure variations, and
estimate the blood pressure by adding a reference blood pressure to the final blood pressure variation.

6. The apparatus of claim 1, wherein the processor is further configured to:
select at least a portion of the plurality of blood pressure estimation models based on the combining coefficient obtained for each of the plurality of blood pressure estimation models,
obtain a final blood pressure variation based on blood pressure variations of the selected portion of the plurality of blood pressure estimation models, and
estimate the blood pressure based on the final blood pressure variation.

7. The apparatus of claim 6, wherein the processor is further configured to select blood pressure estimation models having combining coefficients greater than or equal to a predetermined threshold value.

8. The apparatus of claim 6, wherein the processor is further configured to obtain, as the final blood pressure variation, a statistical value including a mean value or a median value of the blood pressure variations of the selected portion of the plurality of blood pressure estimation models.

9. The apparatus of claim 1, wherein the processor is further configured to:
calculate a statistical value including a mean or standard deviation of the obtained blood pressure variations, and
obtain the combining coefficient for each of the plurality of blood pressure estimation models based on the calculated statistical value.

10. The apparatus of claim 9, wherein the processor is further configured to:
based on the statistical value being greater than a first predetermined value, determine a high combining coefficient for a blood pressure estimation model having a blood pressure variation above a second predetermined value, and
based on the statistical value being less than the first predetermined value, determine a high combining coefficient for a blood pressure estimation model having a blood pressure variation below the second predetermined value.

11. The apparatus of claim 1, wherein the processor is further configured to:
divide a plurality of training data into a plurality of training data groups according to the blood pressure variations, and
generate blood pressure estimation models fir each of the divided training data groups.

12. A method of estimating blood pressure, the method comprising:
measuring a photoplethysmogram (PPG) signal from an object;
obtaining a blood pressure variation for each of a plurality of blood pressure estimation models based on the PPG signal by using the plurality of blood pressure estimation models;
obtaining a combining coefficient for each of the plurality of blood pressure estimation models based on the obtained blood pressure variations; and
estimating blood pressure by using the obtained combining coefficients.

13. The method of claim 12, wherein the obtaining the combining coefficient for each of the plurality of blood pressure estimation models comprises:
obtaining a difference between a reference value and the blood pressure variation for each of the plurality of blood pressure estimation models; and
obtaining the combining coefficient for each of the plurality of blood pressure estimation models based on the obtained difference.

14. The method of claim 13, wherein the obtaining the combining coefficient for each of the plurality of blood pressure estimation models comprises obtaining, as the combining coefficient for each of the plurality of blood pressure estimation models, a value obtained by dividing the difference between the reference value and the blood pressure variation for each of the plurality of blood pressure estimation models by a sum of differences between the reference value and each of the blood pressure variations for the plurality of blood pressure estimation models.

15. The method of claim 12, wherein the estimating the blood pressure comprises:
obtaining a final blood pressure variation by applying the combining coefficient for each of the plurality of blood pressure estimation models to each of the corresponding blood pressure variations and linearly combining the blood pressure variations; and
adding a reference blood pressure to the final blood pressure variation.

16. The method of claim 12, wherein the estimating the blood pressure comprises:
selecting at least a portion of the plurality of blood pressure estimation models based on the combining coefficients obtained for each of the plurality of blood pressure estimation models:
obtaining a final blood pressure variation based on blood pressure variations of the selected portion of the plurality of blood pressure estimation models; and
estimating the blood pressure based on the final blood pressure variation.

17. The method of claim 16, wherein the selecting at least the portion of the plurality of blood pressure estimation models comprises selecting blood pressure estimation models having combining coefficients greater than or equal to a predetermined threshold value.

18. The method of claim 16, wherein the estimating the blood pressure comprises obtaining, as the final blood pressure variation, a statistical value including a mean value or a median value of the blood pressure variations of the selected portion of the plurality of blood pressure estimation models.

19. The method of claim 12, wherein the obtaining the combining coefficient for each of the plurality of blood pressure estimation models comprises:
calculating a statistical value including a mean or standard deviation of the obtained blood pressure variations; and
obtaining the combining coefficient for each of the plurality of blood pressure estimation models based on the calculated statistical value.

20. An electronic device comprising:
a main body;
a photoplethysmogram (PPG) sensor configured to measure a PPG signal from an object; and
a processor disposed in the main body, the processor being configured to:
obtain a blood pressure variation for each of a plurality of blood pressure estimation models based on the PPG signal by using the plurality of blood pressure estimation models,
obtain a combining coefficient for each of the plurality of blood pressure estimation models based on the obtained blood pressure variations, and
estimate blood pressure by using the obtained combining coefficients.
